(19) United States
(12) Patent Application Publication
(10) Pub. No.: US 2010/0137813 A1
(43) Pub. Date: Jun. 3, 2010

(54) LUER CONNECTORS, COMPONENTS THEREOF AND FLUENT MATERIAL DELIVERY DEVICES UTILIZING THE SAME

(75) Inventors: Christopher S. Mudd, Goleta, CA (US); Blanca Guillen, Goleta, CA (US)

Correspondence Address: ALLERGAN, INC., 2525 DUPONT DRIVE, T2-7H, IRVINE, CA, 92612-1599, US

(73) Assignee: ALLERGAN, INC., Irvine, CA (US)

(21) Appl. No.: 12/697,973

(22) Filed: Feb. 1, 2010

Related U.S. Application Data
(63) Continuation of application No. 12/275,548, filed on Nov. 21, 2008.
(60) Provisional application No. 60/991,483, filed on Nov. 30, 2007.

(51) Int. Cl. A61M 5/34

(52) U.S. Cl. 604/241

(57) ABSTRACT

Disclosed herein are luer connectors, components thereof and fluent material delivery devices utilizing the same. The disclosed luer lock connectors can be used with syringe and needle assemblies to resist leakage through and disengagement of the syringe and needle assembly.

### CROSS-REFERENCE TO RELATED APPLICATIONS

This application is a continuation of Ser. No. 12/275,548, filed Nov. 21, 2008, and claims the benefit of U.S. provisional patent application No. 60/991,483 filed Nov. 30, 2007, the entirety of which is hereby incorporated by reference herein.

### FIELD OF THE INVENTION

The present disclosure relates to luer connectors and components thereof as used with fluent material delivery devices such as syringe and needle assemblies. The disclosed luer connectors may include a locking feature to prevent leakage through and disengagement of the syringe and needle assembly.

### BACKGROUND OF THE INVENTION

Luer-type connections have been used for many years in a variety of fluid handling settings, particularly in medical environments. They are used commonly to detachably connect a needle to a syringe to inject medications or to withdraw fluids. Standard luer connectors, as the term is used herein, are characterized by complementary “male” and “female” interlocking parts, commonly used to detachably assemble devices such as syringes, catheters, hubbed needles and intravenous (IV) delivery tubes. Generally, the male and female parts are slightly and complementarily tapered at about six degrees and are assembled with an axial, twisting motion that results in a friction fit.

Certain luer connection systems may include a locking feature in which one of the luer connector elements, usually the male luer element, is surrounded by an internally threaded wall that is engageable by a flange on a hub of the other, usually female, luer element. The lockable components are then assembled by screwing the hub and internally threaded wall together in a secure friction fit while providing some additional resistance to axial separation.

Although the axial, twisting motion or screwing together of the interlocking components can enhance the security of the connection, such connectors may, nevertheless, still leak and disengage during use. The risk of leakage and disengagement are increased with fluid or fluent materials having higher viscosities, increased lubricity effect under pressure, extrusion force, and consequent pressure generated with the syringe barrel and the characteristics of the inner lumen of the needle through which the material is injected. In this higher-pressure situation, fluid may seep between the male and female luer elements and loosen the connection. A loosened connection can result in increased leakage and disengagement. Therefore, there remains a need for improved luer connection systems.

### SUMMARY OF THE INVENTION

The present disclosure provides lure connectors that increase leak resistance and connection security as compared to presently-used luer connectors. The disclosed luer connectors provide these benefits by including a compressible, resilient seal between the interlocking elements. Therefore, when the male and female luer elements are connected, the seal is compressed between them, reducing the risk of leakage and disengagement.

Disclosed herein are luer connectors that have an elastomeric member between the male and female luer elements that increase resistance to leakage and disengagement as compared to presently-used luer connectors. The provided sealing member is compressed to form a secure seal, preventing leakage and decreasing the occurrence of disengagement during use. The annular resilient seal may, for example, be in the form of a washer or an O-ring placed at the bottom of the luer lock socket to be engaged with the end of the flange at the proximal end of the hub as the components are screwed together. The washers or O-rings may instead or in addition to, be disposed about the hub to engage the threads on the internal wall defining the socket. In both cases, the elastomeric element provides additional frictional grip between the hub and the socket and enhances the seal between the two, further reducing risk of separation and preventing leakage forward of the seal.

The luer connector may include a first connector component containing a socket, where the socket includes a base member, a male luer element and an internal wall. The male luer element and wall may extend distally from the base member, with the inner face of the wall surrounding the extending luer element and having threads facing the luer element. The socket may include at least one sealing member to provide a compressible, resilient seal between the hub and the surface of the wall.

The second connector component may include at least one extension adapted to engage the threads of the wall of the first connector component as the male and the female luer elements are mated. The second connector component may effect a threaded connection between the first connector component and the second connector component when luer elements are in a frictional fit, with at least one sealing member compressed between an internal surface of the socket of the connector components, reducing the risk of separation and leakage during use.

The sealing member provides a compressible, resilient seal between the first connector component and the second connector component. The sealing member may include one or more sealing members, such as annular washers or O-rings, disposed within the socket, against the base member or around the male luer element, before coupling of the first connector component and the second connector component. Alternatively, the sealing member may be bonded to the first connector component or mounted on the outside of the second connector component. Furthermore, the wall of the first component may be sufficiently transparent to allow for observation of the sealing member and the resulting connection.

The lure connector may involve a needle assembly for a syringe. In this embodiment, the first connector component may include the distal end of a syringe and the second connector component may include the hub of a needle assembly. The first connector component, containing a male luer element, may be received by the second connector component, containing a female luer element adapted to receive the male luer element in a friction fit. At least one sealing member may be compressed between the connector components, providing extra support to the connection. The sealing member may be disposed within the socket, or attached to the first or second connector components.

Fluent material delivery devices, such as drug delivery devices, may be used deliver a high viscosity fluent material such as dermal filler. Such devices may include a syringe and syringe barrel containing a high viscosity fluent material, a needle assembly, and a sealing member. The linear viscosity of the fluent material being delivered may be at least about 4 Pa, or within the range of about 4 Pa to about 15 Pa. The risk of leakage and disengagement of the needle increases when the contained fluid or fluent being injected has a higher viscosity. The higher extrusion forces and increased lubricity effect result in increased pressure within the syringe, causing a loosened connection, fluid seepage between the components, and eventual disengagement. In accordance with the present invention, fluent delivery devices are provided which comprise, in part, one or more sealing members, for example, having a durometer between about 10 and 90 on the Shore A scale. It has been discovered the use of such sealing members decreases the risk of connector dislodgement when the internal syringe pressure increases, such as to pressures of about 4.5 MPa.

### BRIEF DESCRIPTION OF THE FIGURES

FIG. 1 depicts a prior art syringe and a needle assembly attached by a luer lock connector;

FIG. 2 is an enlarged cross-sectional depiction of the distal end of the prior art syringe depicted in FIG. 1;

FIG. 3. is an enlarged depiction of a prior art needle assembly appropriate for use with a threaded luer connector and the syringe depicted in FIG. 2;

FIG. 4 is a cross-sectional depiction of the prior art needle assembly depicted in FIG. 3;

FIG. 5 depicts a male luer element at the distal end of a syringe with an annular sealing member in accordance with the present invention;

FIG. 6 depicts a needle assembly attached to the male luer connector of FIG. 5;

FIG. 7 depicts another embodiment of the invention wherein the sealing member(s) are provided on the hub of the needle assembly; and

FIG. 8 is a sectional depiction of the needle assembly depicted in FIG. 7 connected to the distal end of a syringe.

### DETAILED DESCRIPTION

Standard luer connectors consist of “male” and “female” interlocking parts that are maintained in connection through a friction fit. Certain luer connectors are provided with a locking feature in which one of the connectors is provided with threads that are engageable by a flange on the other connector. The connectors are assembled by screwing them together to draw the male and female luer components together into a secure friction fit.

Although providing such screwable threads on luer connectors can enhance the security of such connections, these connections nonetheless can leak and disengage. In the case of syringes, the risk of leakage and disengagement is increased when fluids or fluent materials with high viscosities are injected. The increased risk is due to an increased lubricity effect under pressure, higher extrusion forces and the resulting increased pressure generated within the syringe barrel. In this higher-pressure situation, fluid may seep between the surfaces of the male and female luer elements and loosen the connection. A loosened connection can result in increased leakage and eventual disengagement.

Disclosed herein are luer connectors, components thereof and fluent material delivery devices utilizing the same that resist leakage and disengagement of luer connectors. The luer connectors of the present invention achieve this benefit by providing a resilient, compressible sealing member between the luer connector components. When connected, the sealing member is compressed to form a secure seal to prevent leakage and to enhance the security of the threaded connection.

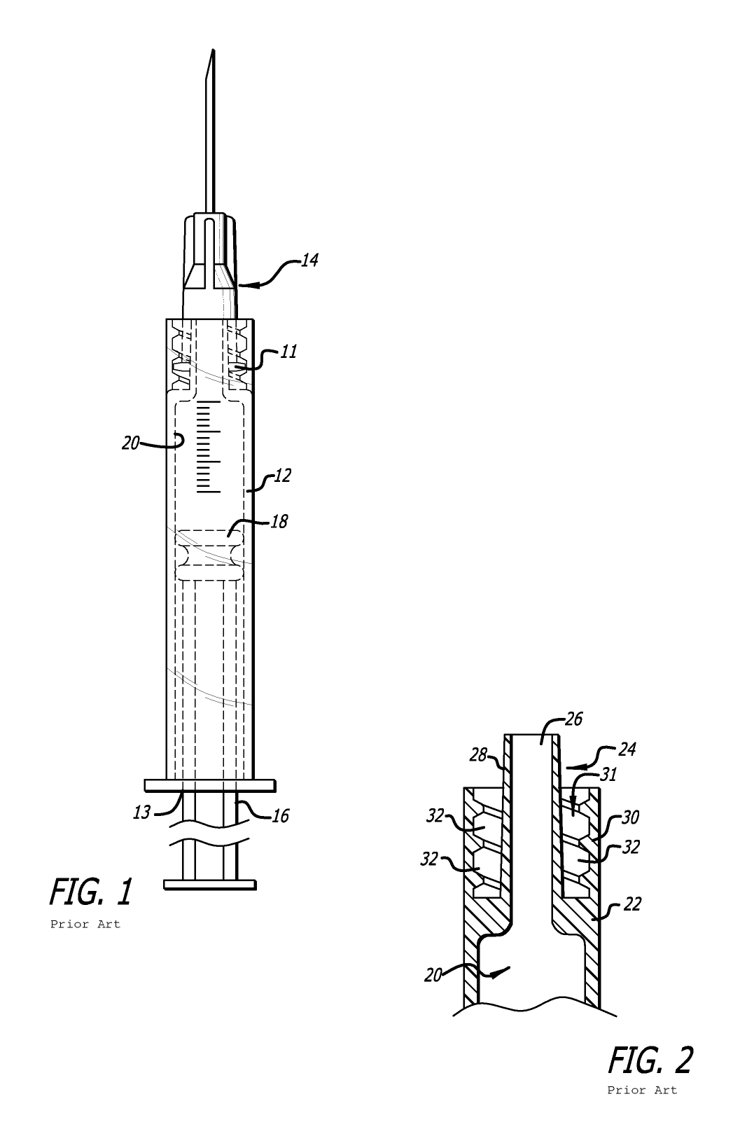

Turning to the figures, FIG. 1 depicts a typical prior art syringe 10 that may be used in a medical or other setting. The syringe 10 has a barrel 12 with a needle assembly 14 detachably connected to its distal end 11. A plunger 16 extends into the barrel of the syringe 10 through an opening at its proximal end 13. The plunger 16 has a stopper 18 that slides along the inner surface of the barrel 12 to define a variable volume internal chamber 20. The plunger 16 may be advanced to eject fluid from the chamber 20 through the needle assembly 14 or may be withdrawn to aspirate fluid through the needle into the chamber 20.

FIG. 2 is an enlarged sectional depiction of the distal end of a prior art syringe. This depiction shows a distal base member 22 and a distally projecting male luer element 24 extending from the base member 22. The male element 24 has a central lumen 26 that communicates with the interior chamber 20. In the depicted embodiment, the outer surface 28 of the male luer element 24 is tapered at a shallow angle, typically about 6°. This tapering can enhance the friction fit connection with a similarly tapered female luer element. To enhance the security of the connection between the male luer element 24 and the female luer element further, a wall 30 may extend distally from the base member 22. The wall 30 may be considered as defining a socket 31 in combination with the male luer element 24 and the base member 22. The wall 30 can be provided with internal threads 32 adapted to engage a needle assembly hub as described below.

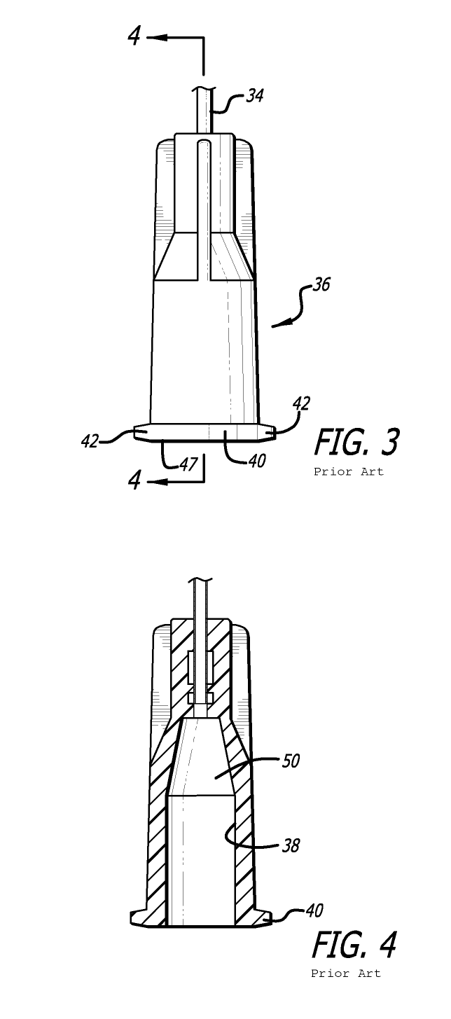

As shown in FIGS. 3 and 4, a prior art needle assembly typically includes a needle 34 securely attached, at its proximal end, to a hub 36. The proximal end 47 of the needle hub 36 is open to expose an axial passageway 38 having a taper 50 and dimensions to form the female luer element adapted to mate with the outer surface of the male luer element. The interior lumen of the needle 34 communicates with the tapered passageway 38 in the hub 36.

The proximal end 47 of the needle hub 36 includes a radially projecting flange 40 and extensions 42. The extensions 42 project out radially more than the intermediate portions of the flange 40 (FIG. 3). The extensions 42 are dimensioned and configured so that when the needle assembly is attached to the distal end of a syringe, the extensions 42 can engage the threads 32 on the inner surface of the wall 30 (FIG. 2) to facilitate screwing the needle assembly (female luer element) to the distal end of the syringe (male luer element). When the syringe and needle assembly are screwed together in this manner, the connection between the female luer element and the male luer element presents a close friction fit that, ideally, would not leak or dislodge.

Notwithstanding the friction fit of these surfaces and the threaded engagement of the male and female luer elements, such connections can leak and in some instances disengage. As stated, this outcome is particularly likely in connection with the injection of high viscosity materials, such as dermal fillers. In such cases, the flow resistance is substantially increased and the clinician must apply substantial force to the plunger in order to inject the material. Such forces increase internal pressure and may cause the luer elements and the connections between them to fail. Additionally, the increased pressure may reduce the viscosity of the material being injected, allowing it to fill minute crevices between the connecting luer elements, further reducing the integrity of the connection.

The present invention provides luer connectors, components thereof and fluent material delivery devices utilizing the disclosed luer connectors, providing increased leak resistance and connection security. The increased leak resistance and connection security are achieved by providing a sealing member between the male and female luer elements. When the male and female luer elements are connected, the sealing member is compressed between them to form a secure seal that helps to prevent leakage and enhances the security of the threaded connection.

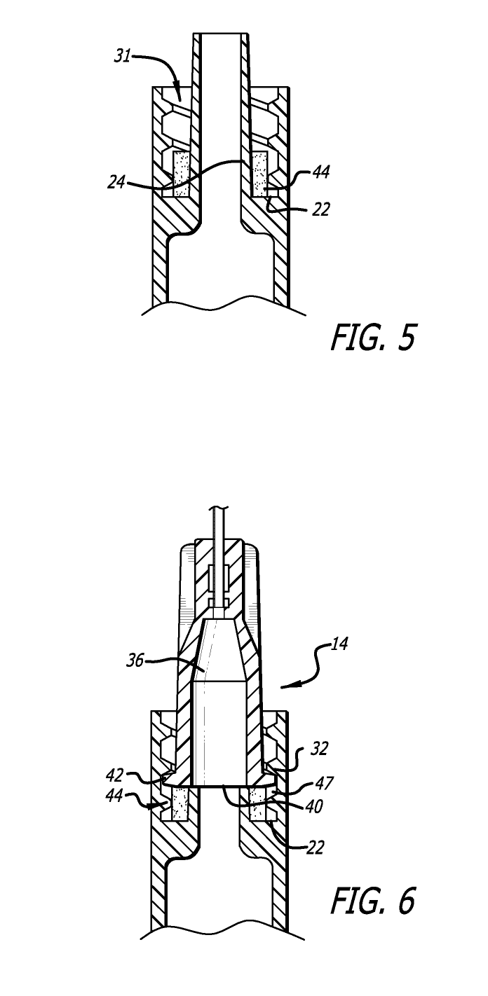

In one embodiment of the invention, shown in FIGS. 5 and 6, the sealing member 44 may include one or more annular washers or O-rings extending around the base of the male luer element 24. These sealing members 44 are in contact with the base member 22 of the annular socket 31.

As shown in FIG. 6, when a needle assembly 14 is attached and screwed together with the distal end of the syringe, the sealing member 44 will be compressed between the proximal end 47 of the needle hub 36 and the base member 22 of the annular socket 31 providing additional leak-resistance between the male and female luer elements. Even if some fluid were to seep between the surfaces of the male and female luer elements, the seal defined by the sealing member 44 would prevent leakage distally beyond that element.

The sealing member 44 will also press the female luer element outwardly against the threads 32 of the annular socket enhancing the frictional fit of the male and female luer elements. That is, when compressed, the resilience of the sealing member 44 biases the extensions on the needle assembly hub more firmly against their engaging surfaces of the internal threads 32 to urge them together more firmly. This additional pressure provides additional resistance to disengagement.

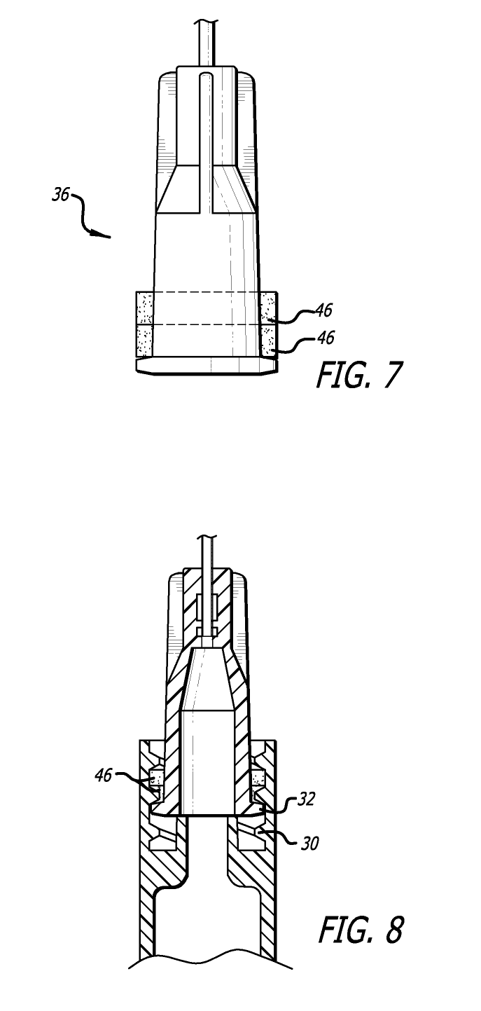

FIGS. 7 and 8 depict another embodiment of the luer connectors of the present invention. In this depicted embodiment, two sealing members 46 are disposed about the exterior of the needle assembly hub 36. As will be understood by one of ordinary skill in the art, one or more than two sealing members could also be used. When the needle assembly is engaged with the male luer element, the sealing member 46 engages the threads 32 on the inner surface of the wall 30 to resist leakage and dislodgement.

The disclosed sealing members can be formed from a resilient, compressible and/or elastomeric material. Appropriate elastomeric materials include, without limitation, Buna-N (Nitrile) (NBR), propylene (EPDM), silicone (MQ; PMQ; VMQ; PVMQ), cast polyurethane, Chloroprene (Neoprene®, DuPont Performance Elastomers), Fluorocarbon (Viton®, DuPont Performance Elastomers), (Fluorel®, 3M Corporation) (FKM), Fluorosilicone (FVMQ) or silicone rubber, all available from Apple Rubber Products, Inc.

The sealing members may be formed from elastomeric materials with a durometer in the range of about Shore A 10 to about Shore A 90. The sealing members can also be selected to have a relatively high coefficient of friction with respect to the syringe and/or needle assembly surfaces with which they are in contact. A high coefficient of friction with these surfaces provides an additional measure of resistance to disengagement. In general, it should be understood that the dimensions and number of sealing members may be selected as desired to provide the desired levels of sealing and frictional characteristics.

As previously stated, the luer connectors disclosed herein are particularly useful for the injection of highly viscous substances. By way of non-limiting example, the luer connectors are particularly useful with fluent materials having linear viscosities, at room temperature in the range of about 4 Pa to about 15 Pa.

One particularly useful application of the disclosed luer connectors is for the administration of dermal fillers used to cosmetically reduce skin wrinkles by injecting the filler subcutaneously beneath the wrinkled regions. Typically, such procedures involve the use of fine gauge needles, for cosmetic purposes, that increase the flow resistance of the viscous material. Consequently, the clinician typically must apply substantial force to the plunger of the syringe, of the order of about 25 to about 75 newtons, to inject the material. Such forces generate high internal pressures such as about 0.9 MPa to about 4.5 MPa. Without wishing to be bound by any particular theory of operation, it is believed that this amount of pressure tends to reduce the viscosity of the injected material such that it can be forced into minute crevices between the mating surfaces of the male and female luer elements.

Dermal fillers appropriate for application with the disclosed luer connectors include, without limitation, Juvederm® (Allergan, Inc.), Restylane® (AB Corp.), Artefill®, (Artes Medical, Inc.), Captique® (Genzyme Corp.), Cosmoplast® (Inamed Corp.), Cosmoderm® (Inamed Corp.), Hylaform® (Biomatrix, Inc.), Perlane® (AB Corp.), Radiesse® (Bioform Medical, Inc.) and Sculptra® (Aventis, LLC).

Pre-packaged fluent material delivery devices utilizing the disclosed luer connectors and components thereof are also provided. In one embodiment, the fluent material delivery device is a syringe provided pre-filled with a fluent material of choice that can include, but is not limited to, the dermal fillers described above. The fluent material can also be any drug or other bioactive material appropriate for administration through a syringe. The syringe can be provided pre-assembled with its needle assembly in place or a clinician can connect the syringe and needle assembly before use. When a syringe is provided pre-filled with a fluent material of choice, it is intended to be a single-use syringe. Other syringes adopting the luer connectors described herein can be multi-use syringes.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A luer connector for a syringe, the luer connector comprising:
a first connector component including a male luer element, a base portion and a wall, the base portion circumscribing the male luer element and the wall circumscribing the base portion;
a second connector component including a female luer element having a proximal end and adapted to receive said male luer element of said first connector component; and
a resilient sealing member compressed between the base portion of the male luer element and the proximal end of the female luer element, the sealing member biasing the second connector component into engagement with the first connector component.

2. The luer connector according to claim 1 wherein the wall of said male luer element includes threads.

3. The luer connector according to claim 2 wherein the proximal end of the female luer element includes a lip structured to engage the threads of the wall of the male luer element.

4. The luer connector according to claim 3 wherein said sealing member is compressed between the lip of the female luer element and the threads of the wall of the male luer element.

5. The luer connector according to claim 1 wherein said first connector component comprises the distal end of a syringe and said second connector component comprises a hub of a needle assembly.

6. The luer connector according to claim 1 comprising a plurality of resilient sealing members.

7. The luer connector according to claim 1 wherein said sealing member has a durometer of between about 10 and about 90 on the Shore A scale.

8. The luer connector according to claim 1 wherein said sealing member resists dislodgement of said first connector component and said second connector component up to pressures in said syringe of about 4.5 MPa.

9. A delivery device for a high viscosity fluent material comprising:
a syringe/needle assembly comprising
a syringe including a male luer element, a base portion and a wall, the base portion circumscribing the male luer element and the wall circumscribing the base portion,
a needle assembly including a female luer element having a proximal end and adapted to receive said male luer element of said first connector component, and
a resilient sealing member compressed between the base portion of the male luer element and the proximal end of the female luer element, the sealing member biasing the second connector component into engagement with the first connector component; and
a high viscosity fluent material contained in the syringe.

10. The device of claim 9 wherein said sealing member resists dislodgement of said first connector component and said second connector component up to pressures in said syringe of about 4.5 MPa.

11. The device according to claim 9 wherein said sealing member has a durometer of between about 10 and about 90 on the Shore A scale.

12. The device according to claim 9 wherein said fluent material comprises a dermal filler.

13. The device according to claim 12 wherein the linear viscosity of said fluent material is at least about 4 Pa.
